(19) United States
(12) Patent Application Publication
(10) Pub. No.: US 2011/0060377 A1
(43) Pub. Date: Mar. 10, 2011

(54) MEDICAL CO-PROCESSOR FOR SIGNALING PATTERN DECODING AND MANIPULATION OF CELLULAR STRUCTURES

(75) Inventor: Newton Howard, Arlington, VA (US)

(73) Assignee: MAKE A MIND CO., INC., Cambridge, MA (US)

(21) Appl. No.: 12/880,042

(22) Filed: Sep. 10, 2010

Related U.S. Application Data
(60) Provisional application No. 61/241,314, filed on Sep. 10, 2009.

(51) Int. Cl. A61N 1/00; A61B 5/0476

(52) U.S. Cl. 607/3; 600/544

(57) ABSTRACT

Devices, methods and systems for affecting living tissues (brain, spinal stem) by neuronal querying, decoding responses, determining signaling patterns for direct delivery of a range of signals to the neural tissue in order to induce specific cell behaviors through intrusive and non-intrusive methods.

### CROSS-REFERENCE TO RELATED APPLICATIONS

This application claims the benefit of Provisional Application No. 61/241,314, filed Sep. 10, 2009, the contents of which are incorporated herein in their entirety.

### BACKGROUND OF THE INVENTION

1. Field of the Invention

The present invention relates to devices, methods, and systems for affecting living tissues (brain, spinal stem) by neuronal querying, decoding responses, determining signaling patterns for direct delivery of a range of signals to the neural tissue in order to induce specific cell behaviors through intrusive and non-intrusive methods.

2. Description of the Related Art

Mental states are the manifestations of particular neural patterns firing and neurotransmitters exchanged between neurons. These states have neural correlations corresponding to specific electrical circuits. A decade ago there was a deep interest in functional neurosurgery for neural disorders, such as movement disorders. This led to an increase in understanding of the underlying neural mechanisms and circuitry involved in basal ganglia disorders with improved surgical techniques and the development of deep brain stimulation (DBS) technology which paved the way for major advances in the treatment of Parkinson's Disease (PD) and other neurological disorders.

To better understand the role of the posterior parietal cortex, basal ganglia and cerebellum in the control of movement, scientists inserted electrodes into patients with movement disorders such as Parkinson's disease (PD). These electrodes helped stimulate the control network system (CNS) for which low frequency (4-15 Hz) field potentials were recorded that correlated with the patient's involuntary movements. Interestingly, recent studies have discovered that the pedunculopontine nucleus (PPN) in the upper brainstem has extensive connections with several motor centers in the CNS [Nandi, 2002] and is very important in controlling proximal muscles for posture and locomotion.

This area is over-inhibited in many patients, which is a major cause of their inability to move, i.e. in an akinesia state. This inhibition can be overcome by stimulating the PPN directly and can thus return previously chair-bound patients to a useful life. That is why, Deep brain stimulation (DBS) of the pedunculopontine nucleus (PPN) is a novel neurosurgical therapy developed to address symptoms of gait freezing and postural instability in Parkinson's disease and related disorders. [Aziz, 2008]

Neuropathic pain arises from damaged neural tissues which can be essential when the neural injury is in the brain or spinal cord. In patients with intractable central neuropathic pain the pain seems to be caused by spontaneous oscillations in the ‘central pain matrix’ which consists of the periaqueductal gray, pen-ventricular gray (PAG/PVG), globus pallidus, thalamus, anterior cingulate, insula and the orbitofrontal cortex. It was found that driving the PAG/PVG by stimulating at 10 Hz, one can eliminate the oscillations and reduce the patients' feelings of pain very considerably. [Stein] Pain suppression is frequency dependent and pain relief occurred at PVG simulation levels ranging from 5-25 Hz. There are also correlations between thalamic activity and chronic pain. This low frequency potential may provide an objective index for quantifying chronic pain, and may hold further clues to the mechanism of action of PVG stimulation. [Liu, 2002]

While is has been widely discussed that specific frequencies affect neural tissue functioning and development, the mechanisms guiding this effect have not been found. Understanding how frequencies affect the complex electrochemical structures and processes in neural tissue, and being able to determine the ranges and sequences that aid and/or restore normal neural development and activity, are seen as the next step in addressing neurological disorders. Furthermore, non-neural cells are driven by electrochemical processes and can be subjected to similar treatments.

### SUMMARY OF THE INVENTION

A co-processor, using brain stimulation and feedback methods, provides a series of signals to the neural tissue and analyzes the response signals using pre-determined or dynamically determined signatures, thereby decoding the patient-, tissue- and -disorder-specific cellular structure signals. The co-processor then chooses ranges and sequences of treatment frequencies and sends modulated signals to the targeted tissue via a variety of effector devices such as enzymic controllers, optogenetic interfaces, and other signal carrier techniques to stimulate the cells for specific protein switching/folding or electrochemical signaling sequences. The modulated signals therefore address cell disorders by targeted signaling which activates the cells' internal resources on a level deeper than existing treatments.

### BRIEF DESCRIPTION OF THE DRAWINGS

FIG. 1 is a high-level representation of a coprocessor determining specific signal patterns and producing signals affecting cell functioning by intrusive and non-intrusive stimulation of target tissue.

FIG. 2 is a high-level representation of coprocessor functions for implementing the manipulation of cellular structures via signaling, as outlined in FIG. 1.

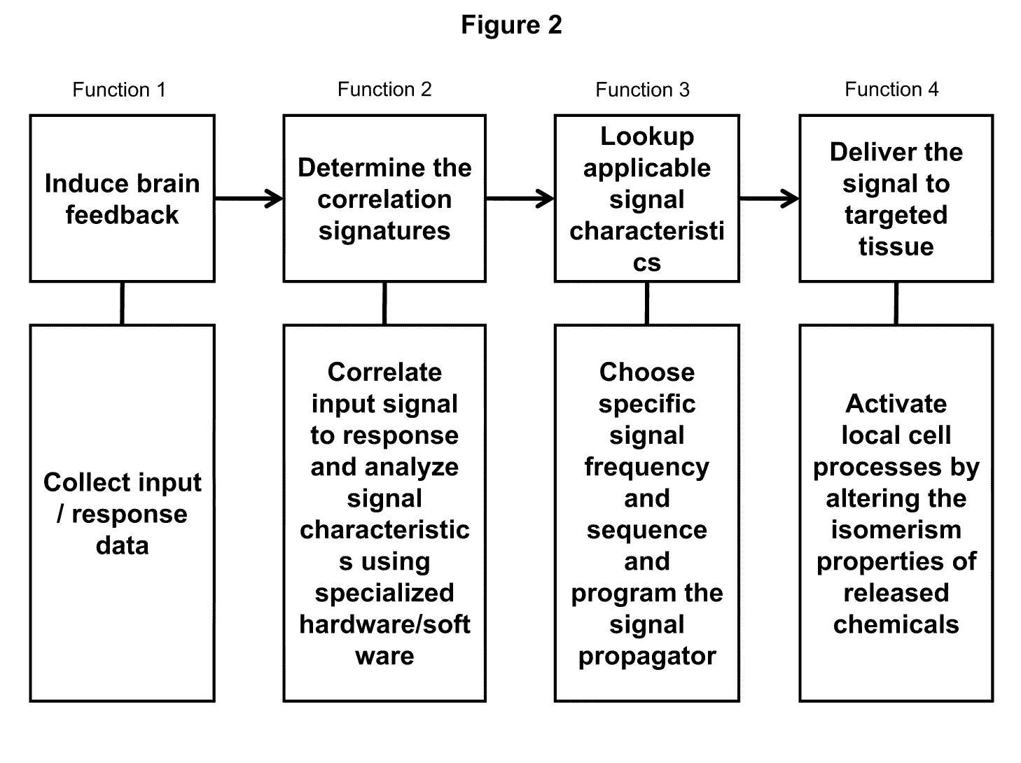

FIG. 3 is an example of an apparatus implementing the invention, demonstrating the interconnections and functions of its composite parts.

FIG. 4 is an exemplary block diagram of a computer system 200, which may be used to implement an analyzer.

### DETAILED DESCRIPTION OF THE INVENTION

Thoroughly examining brain signaling information exchange from high-level cognitive functions down to the molecular interactions, it is possible to mathematically represent these processes in a uniform manner There exists a set of functions, which together with the unary construct form the new system of mathematics—the unitary system. The brain communicates with other organs, and within itself, in unitary operators. The unitary operators carry a state of time and space, conveying all that is necessary to decipher any semantic- or non-semantic-based language, including pictorial languages, and down to the level of single neuron signaling.

There is a connection between that unitary system and the neurological functions of the units of the brain, neurons—starting with the action potential, down to the stimuli effect and further down to the cellular level in the body, including the DNA encoding and decoding specific protein segments.

The frequency oscillations are the underlying cause of most of brain disorders, and it is important to understand the nature of such frequencies, their causes, their ranges, and the relation of each such range to each disorder. There exists a prime relation between optical (or other signal) frequency and that of electrochemical releases in the cells and the properties of chemicals, most important of which is the choice of optical isomers of the chemicals. The relation is governed by the rules of optics and electro physics. This fundamental relation has applications for diagnostics and treatment of neural disorders, starting with depression, and has wide implications for understanding of cognitive processes.

What we propose is an apparatus using this core relation for affecting living tissues (e.g., brain, spinal stem) by neuronal querying/feedback, decoding responses, determining signaling, and delivering a of a range of signals to the neural tissue in order to induce specific cell behaviors, through intrusive and non-intrusive methods.

An example embodiment of the invention can be described as follows:

An (1) Analyzer comprised of commodity hardware parts, whose primary purpose is to provide data look-ups in a pre-loaded database of tissue/frequency/isomer N-tuples, and a (2) Signal Propagator, reconfigurable at runtime to efficiently deliver high-frequency signal sequences via a variety of carriers.

The Analyzer is connected via Interface 1 to an array of probes. The probes are used to examine areas of brain tissues, collect the frequencies of neuronal activity, and transmit these frequencies to the Analyzer for processing. The probes can be invasive, e.g., implantable electrodes, or using non-intrusive methods, such as fMRI.

Interface 2 connects the Analyzer to a Signal Propagator. The Signal Propagator is used to deliver a range of signals to the targeted neural tissue, in order to induce and guide electrochemical activity. The Signal Propagator is connected to a set of devices which can deliver a modulated signal to the targeted neural tissue via invasive (e g , implanted optical probes) or non-invasive methods (e.g., transcranial stimulation).

The Analyzer, via Interface 2, produces a series of high frequency impulses into the patient's brain tissue to induce neuronal activity feedback, which is collected by Interface 1 as a series of action potential spikes. The Analyzer, using this input, isolates a set of baseband oscillation frequencies specific to the area of activity. These frequencies are correlated by the Analyzer to a set of electro-chemical release sequences which are required to be triggered in the cells to induce specific protein switching/folding sequences. The chemical release sequences are generated by the signals determined by the Analyzer. The Analyzer dynamically reconfigures the Signal Propagator to produce the required sequences of signals, which are delivered, via Interface 2, to the targeted tissue. The signals activate the release of a specific set of positive (+) or negative (−) optical isomers of chemicals in the tissue. The chemical communications triggered by the isomer release activates tissue changes (e.g., re-growth) in the targeted area.

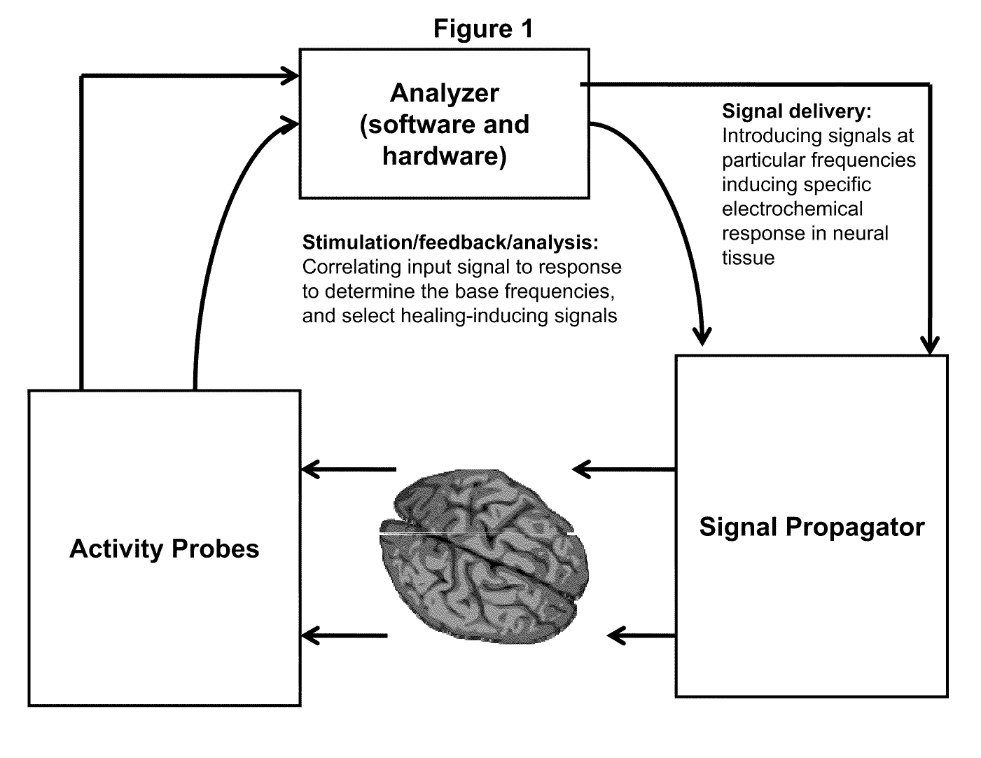

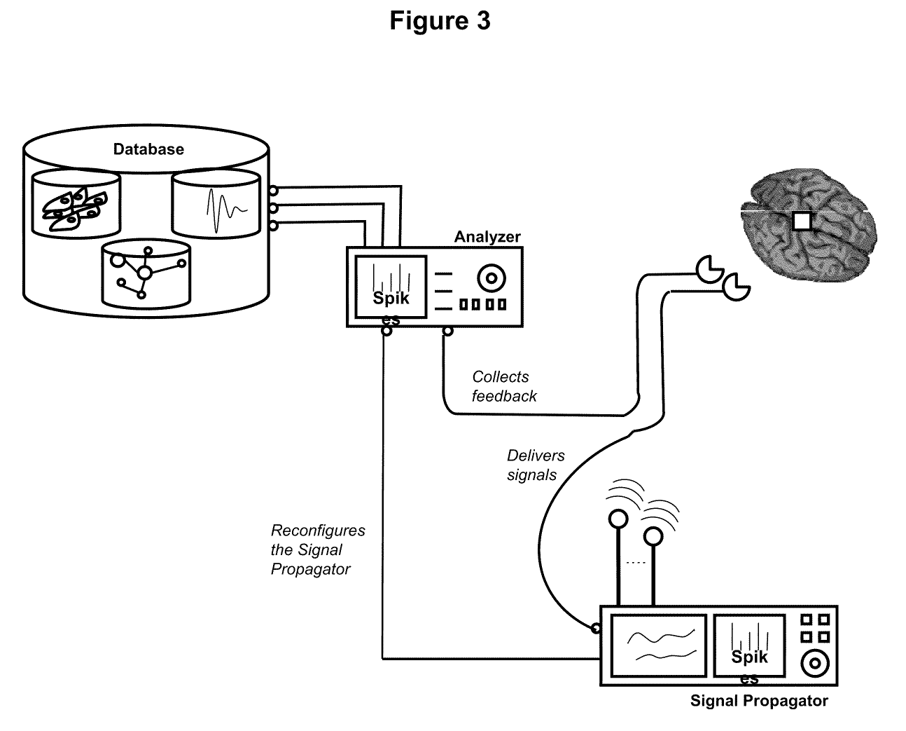

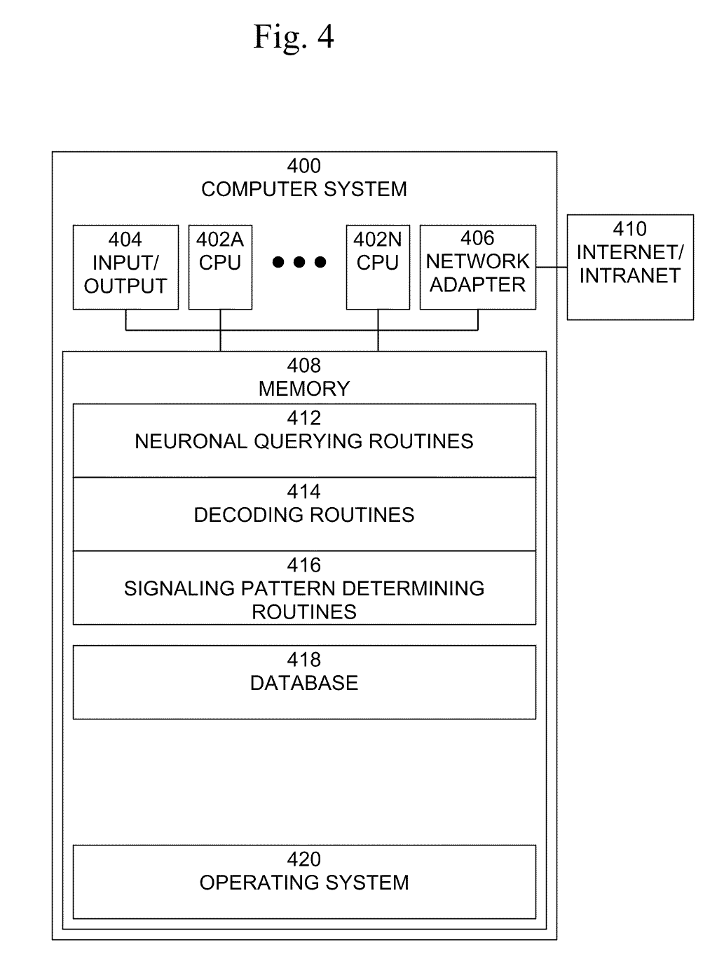

An exemplary block diagram of a computer system 400, such as may be used to implement an analyzer shown in FIG. 1, and/or a database, shown in FIG. 3, is shown in FIG. 4. System 400 is typically a programmed general-purpose computer system, such as a personal computer, workstation, server system, and minicomputer or mainframe computer. System 400 includes one or more processors (CPUs) 402A-402N, input/output circuitry 404, network adapter 406, and memory 408. CPUs 402A-402N execute program instructions in order to carry out the functions of the present invention. Typically, CPUs 402A-402N are one or more microprocessors, such as an INTEL PENTIUM® processor. FIG. 4 illustrates an embodiment in which System 400 is implemented as a single multi-processor computer system, in which multiple processors 402A-402N share system resources, such as memory 408, input/output circuitry 404, and network adapter 406. However, the present invention also contemplates embodiments in which System 400 is implemented as a plurality of networked computer systems, which may be single-processor computer systems, multi-processor computer systems, or a mix thereof.

Input/output circuitry 404 provides the capability to input data to, or output data from, database/System 400. For example, input/output circuitry may include input devices, such as keyboards, mice, touchpads, trackballs, scanners, etc., output devices, such as video adapters, monitors, printers, etc., and input/output devices, such as, modems, etc. Network adapter 406 interfaces database/System 400 with Internet/intranet 410. Internet/intranet 410 may include one or more standard local area network (LAN) or wide area network (WAN), such as Ethernet, Token Ring, the Internet, or a private or proprietary LAN/WAN.

Memory 408 stores program instructions that are executed by, and data that are used and processed by, CPU 402 to perform the functions of system 400. Memory 408 may include electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra direct memory access (UDMA), or a small computer system interface (SCSI) based interface, or a variation or enhancement thereof, such as fast-SCSI, wide-SCSI, fast and wide-SCSI, etc, or a fiber channel-arbitrated loop (FC-AL) interface.

The contents of memory 408 varies depending upon the function that system 400 is programmed to perform. One of skill in the art would recognize that these functions, along with the memory contents related to those functions, may be included on one system, or may be distributed among a plurality of systems, based on well-known engineering considerations. The present invention contemplates any and all such arrangements.

In the example shown in FIG. 4, memory 408 includes neuronal querying routines 412, decoding routines 414, signaling pattern determining routines 416, database 418, and operating system 420. Neuronal querying routines 412 include software that implements neuronal querying of neural tissue, as shown in FIG. 1. Decoding routines 414 include software that implements decoding responses of the neuronal querying, such as by receiving signals in response to the neuronal querying and analyzing the response signals using pre-determined or dynamically determined signatures to decode patient, tissue, or disorder specific cellular structure signals, as shown in FIG. 1. Signaling pattern determining routines 416 includes software that implements determining signaling patterns for direct delivery of signals to the neural tissue in order to induce specific cell behaviors through intrusive or non-intrusive methods, based on the decoded responses of the neuronal querying, such as by choosing ranges and sequences of treatment frequencies and sending signals modulated based on the chosen ranges and sequences of treatment frequencies to the neural tissue via at least one effector device, as shown in FIG. 1. Database 418 stores and provides access to data used in performing the processes described above, such as data including tissue/frequency/isomer N-tuples. Operating system 420 provides overall system functionality.

As shown in FIG. 4, the present invention contemplates implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including UNIX®, OS/2®, and WINDOWS®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

It is important to note that while the present invention has been described in the context of a fully functioning data processing system, those of ordinary skill in the art will appreciate that the processes of the present invention are capable of being distributed in the form of a computer readable medium of instructions and a variety of forms and that the present invention applies equally regardless of the particular type of signal bearing media actually used to carry out the distribution. Examples of non-transitory computer readable media include storage media, examples of which include, but are not limited to, floppy disks, hard disk drives, CD-ROMs, DVDROMs, RAM, and, flash memory.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A method for affecting living tissues comprising:
performing neuronal querying on neural tissue;
decoding responses of the neuronal querying; and
determining signaling patterns for direct delivery of signals to the neural tissue in order to induce specific cell behaviors through intrusive or non-intrusive methods, based on the decoded responses of the neuronal querying.

2. The method of claim 1, wherein the neuronal querying is performed using brain stimulation and feedback methods to provide a series of signals to the neural tissue.

3. The method of claim 1, wherein the decoding is performed by:
receiving signals in response to the neuronal querying; and
analyzing the response signals using pre-determined or dynamically determined signatures to decode patient, tissue, or disorder specific cellular structure signals.

4. The method of claim 1, wherein the signaling patterns are determined by:
choosing ranges and sequences of treatment frequencies; and
sending signals modulated based on the chosen ranges and sequences of treatment frequencies to the neural tissue via at least one effector device.

5. The method of claim 4, wherein the least one effector device comprises an enzymic controller, an optogenetic interface, or a device employing signal carrier techniques.

6. The method of claim 4, wherein the modulated signal stimulate cells of the neural tissue for specific protein switching/folding or electrochemical signaling sequences.

7. A system for affecting living tissues comprising:
a processor operable to execute computer program instructions;
a memory operable to store computer program instructions executable by the processor; and
computer program instructions stored in the memory and executable to perform the steps of:
performing neuronal querying on neural tissue;
decoding responses of the neuronal querying; and
determining signaling patterns for direct delivery of signals to the neural tissue in order to induce specific cell behaviors through intrusive or non-intrusive methods, based on the decoded responses of the neuronal querying.

8. The system of claim 7, wherein the neuronal querying is performed using brain stimulation and feedback methods to provide a series of signals to the neural tissue.

9. The system of claim 7, wherein the decoding is performed by:
receiving signals in response to the neuronal querying; and
analyzing the response signals using pre-determined or dynamically determined signatures to decode patient, tissue, or disorder specific cellular structure signals.

10. The system of claim 7, wherein the signaling patterns are determined by:
choosing ranges and sequences of treatment frequencies; and
sending signals modulated based on the chosen ranges and sequences of treatment frequencies to the neural tissue via at least one effector device.

11. The system of claim 10, wherein the least one effector device comprises an enzymic controller, an optogenetic interface, or a device employing signal carrier techniques.

12. The system of claim 10, wherein the modulated signal stimulate cells of the neural tissue for specific protein switching/folding or electrochemical signaling sequences.

13. A computer program product for affecting living tissues, implemented in a computer system, comprising:
a non-transitory computer readable medium;
computer program instructions, recorded on the non-transitory computer readable medium, executable by a processor, for performing the steps of:
performing neuronal querying on neural tissue;
decoding responses of the neuronal querying; and
determining signaling patterns for direct delivery of signals to the neural tissue in order to induce specific cell behaviors through intrusive or non-intrusive methods, based on the decoded responses of the neuronal querying.

14. The computer program product of claim 13, wherein the neuronal querying is performed using brain stimulation and feedback methods to provide a series of signals to the neural tissue.

15. The computer program product of claim 13, wherein the decoding is performed by:
receiving signals in response to the neuronal querying; and
analyzing the response signals using pre-determined or dynamically determined signatures to decode patient, tissue, or disorder specific cellular structure signals.

16. The computer program product of claim 13, wherein the signaling patterns are determined by:
choosing ranges and sequences of treatment frequencies; and
sending signals modulated based on the chosen ranges and sequences of treatment frequencies to the neural tissue via at least one effector device.

17. The computer program product of claim 16, wherein the least one effector device comprises an enzymic controller, an optogenetic interface, or a device employing signal carrier techniques.

18. The computer program product of claim 16, wherein the modulated signal stimulate cells of the neural tissue for specific protein switching/folding or electrochemical signaling sequences.
